(19) United States
(12) Patent Application Publication
(10) Pub. No.: US 2010/0106052 A1
(43) Pub. Date: Apr. 29, 2010

(54) SURGICAL RETRACTOR

(76) Inventors: Margaret Uznanski, Great Neck, NY (US); Anthony L. Ceniccola, Hamden, CT (US)

Correspondence Address: Tyco Healthcare Group LP, 60 MIDDLETOWN AVENUE, NORTH HAVEN, CT, 06473, US

(21) Appl. No.: 12/578,633

(22) Filed: Oct. 14, 2009

Related U.S. Application Data
(60) Provisional application No. 61/107,743, filed on Oct. 23, 2008.

(51) Int. Cl. A61B 1/32; A61B 10/02

(52) U.S. Cl. 600/562; 600/205

(57) ABSTRACT

A surgical device having a housing, an elongated shaft extending distally from the housing, a retractor including a plurality of retractor elements extending distally from the shaft, a vacuum chamber in communication with a vacuum tube positioned within the elongated shaft for applying a suction force to remove a specimen, and a fluid delivery assembly including a fluid container positioned adjacent the housing.

This application claims priority from U.S. Provisional Application No. 61/107,743, filed Oct. 23, 2008, the entire contents of which are incorporated herein by reference.

### BACKGROUND

1. Technical Field

This application relates to a device for minimally invasive surgery and more particularly to a multi-functional device for minimally invasive thoracic surgery.

2. Description of the Related Art

In recent years, new methods of access into the thoracic cavity have been developed in order to perform some of the surgical procedures done before through a median sternotomy. One minimally invasive method is called a mini-thoracotomy and involves access through an incision running intercostally (between two ribs) of the left chest wall. A surgical retractor is used as for the median sternotomy, but in this case, the superior and inferior rib cages of the left chest are spread apart about two inches, thus resulting in much less overall trauma to the bones, muscles, and other tissues in the chest. Subsequently, the patient endures less pain and irritation following the surgery, and the recovery time is significantly decreased.

The mini-thoracotomy method of access to the thoracic cavity, however, has propagated the need for new surgical tools and methods because the opening into the thoracic cavity is considerably smaller than for the sternotomy. Also, since the internal mammary arteries are attached to the thoracic cavity wall, the angle of approach the surgeon must use through the opening is very difficult since the inferior rib cage tends to obstruct the manipulation of surgical devices used for the procedure.

It would be advantageous to provide a device capable of performing a number of different functions to reduce the number of instrument exchanges and number of access ports to perform a minimally invasive surgical procedure such as in the thoracic cavity.

### SUMMARY

The present disclosure provides in one aspect a multi-functional surgical device comprising a housing, an elongated shaft extending distally from the housing, a retractor including a plurality of elongated retractor members extending distally from the shaft, a vacuum tube positioned within the elongated shaft, a vacuum chamber in communication with the vacuum tube for applying a suction force to remove a specimen from a patient's body, and a fluid container to deliver a fluid through the device into the body.

In one embodiment, at least one of the retractor members includes a lumen extending therethrough, the lumen communicating with the fluid container to apply a fluid to a surgical site. The lumen of the retractor members can have a distal opening. The device can include a web member extending between at least two of the retractor members.

In one embodiment, the fluid container is positioned external of the housing. In one embodiment, the vacuum chamber is positioned inside of the housing. The device can further include a specimen container in communication with the vacuum tube to receive a specimen removed from the body via suction. The specimen container can be positioned external of the housing.

The device preferably includes an actuator for moving the retractor members distally from a first collapsed position within the elongated shaft to a second expanded position distal of the shaft.

In one embodiment, the actuator includes a pusher tube, wherein the vacuum tube is positioned within the pusher tube, and the pusher tube includes a plurality of lumens communicating with the fluid container to deliver a fluid to the lumen of the retractor members.

In one embodiment, one or more of the retractor members includes a sensor.

In another aspect, the present disclosure provides a surgical device comprising a housing, an elongated shaft extending distally from the housing, a vacuum tube positioned within the elongated shaft, a vacuum chamber in communication with the vacuum tube for applying a suction force to remove a specimen from a patient's body, and a fluid container to deliver a fluid to the body. The vacuum chamber is positioned inside of the housing, and a specimen container to receive a specimen removed from the body is in communication with the vacuum tube.

### BRIEF DESCRIPTION OF THE DRAWINGS

Various embodiments view of the present disclosure are described hereinbelow with reference to the drawings, wherein

FIG. 1 is a perspective of the multi-functional surgical device of the present disclosure;

FIG. 2 is an exploded view of the device of FIG. 1;

FIG. 3 is a cross-sectional view of the device of FIG. 1 with the retractor in the undeployed position;

FIG. 4 is a transverse cross-sectional view taken along line 4-4 of FIG. 3;

FIG. 5 is a perspective view similar to FIG. 1 showing the retractor in the deployed position;

FIG. 6 is a longitudinal cross-sectional view taken along line 6-6 of FIG. 5;

FIG. 7 is a close up perspective view of the deployed retractor of the device of FIG. 6; and

FIG. 8 is a perspective view illustrating insertion of the device between a patient's rib in a minimally invasive thoracic procedure.

### DETAILED DESCRIPTION OF THE EMBODIMENTS

The present embodiments of the presently disclosed device will now be described in detail with reference to the foregoing figures wherein like reference numerals identify similar or identical elements. In the figures, and in the description which follows, the term “proximal”, as is traditional, will refer to the end of the device that is closer to the clinician, while the term “distal” will refer to the end of the device that is further from the clinician.

The device of the present disclosure provides a multi-functional surgical device designed for thoracic surgery, although use in other surgical procedures, including other minimally invasive procedures, is also contemplated. The multi-functional surgical device provides controlled tension during delicate dissection, enables manipulation of body tissue, provides for vacuum assisted specimen removal, and provides for fluid delivery to the surgical site.

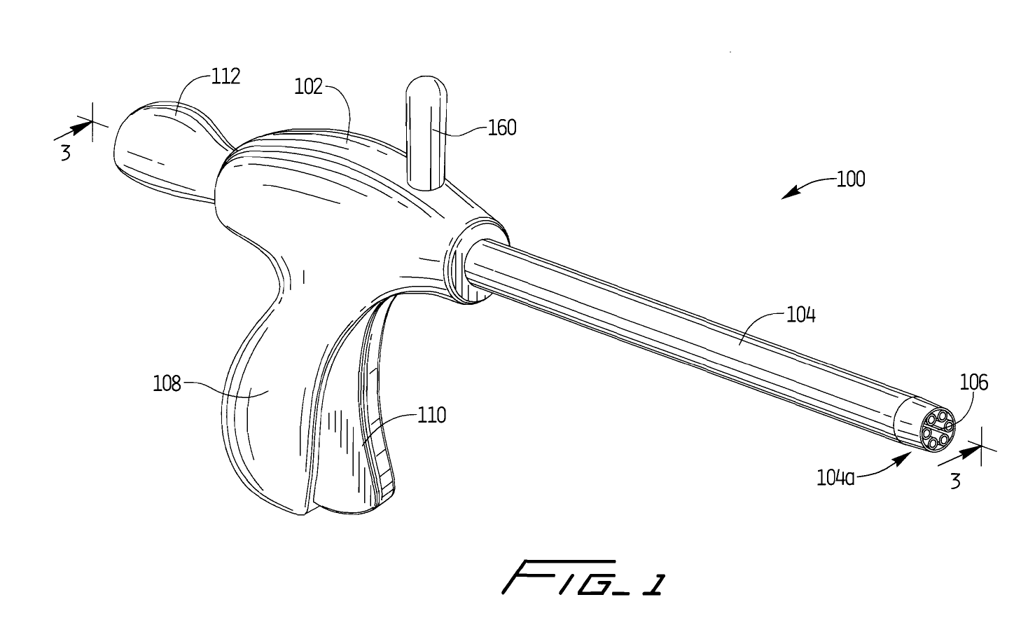

As seen in FIGS. 1-3, one embodiment of the surgical device is disclosed and designated generally by reference numeral 100. Device 100 comprises a housing 102 having a tank or container 112, a fluid container 160, a vacuum chamber 114 positioned within housing 102, and a stationary handle portion 108 extending from the housing 102. An elongated outer tube or shaft 104 extends distally from the housing 102. A retractor 106 is advanceable from the distal end 104a of the tube 104. The handle 108 has an actuator handle 110 pivotally mounted thereto for advancing and deploying the retractor assembly 106 from the elongated tube 104 as described in more detail below.

An articulating mechanism can be provided to change the angular position of the retractor 106 relative to the longitudinal axis of the shaft 104. A separate handle can be provided with an associated linkage or cable mechanism which articulates the retractor to change its angular position. Alternatively, actuator 110 can be configured to articulate the retractor into various grasping/retracting orientations to move or capture specimens.

In an alternative embodiment, the retractor 106 can be positioned distal of the elongated tube 104 in a fixed axial position and configured and dimensioned to collapse as it is inserted through an access port, e.g. a thoracoport (not shown). In such embodiment, once advanced past the distal end of the port, the retractor, which can be normally biased to an open position, would expand to its normal position inside the body cavity. An actuator handle similar to handle 110 (or other type handles or articulation controls) can then be configured to articulate the retractor.

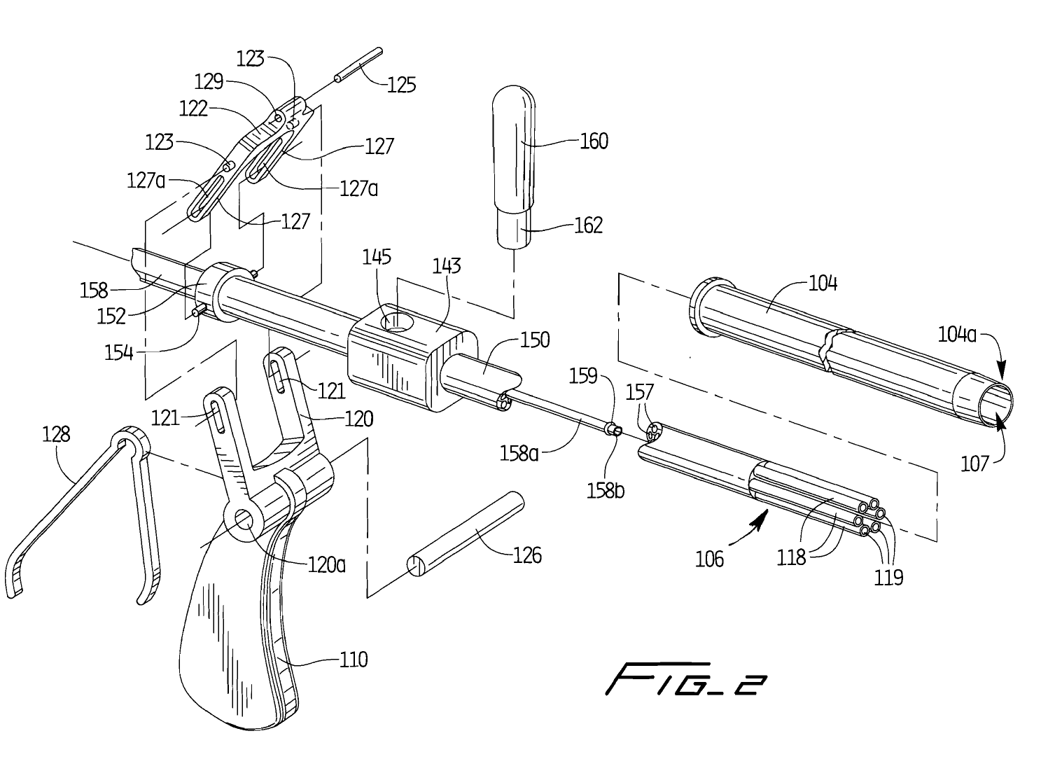

The mechanism for deploying the retractor 106 will now be described with reference to FIGS. 2-4. The deployment mechanism includes an actuator handle 110 having a yoke 120 with a pair of elongated slots 121 formed therein. A link 122 has an elongated slot 127a formed in each arm 127 and a transverse pin 123 extending outwardly from each arm. A connecting pin 125 extends through aperture 129 in link 122 to enable pivotable movement of the link 122 with respect to the housing 102, as the pin 125 is mounted to the housing 102. Spring 128 biases the actuator 110 to the distal position which in turn maintains the retractor 106 within a lumen 141 of tube 104, constrained in the undeployed collapsed position by the wall of the tube 104. Pivot pin 126, mounted to the housing 102, extends through aperture 120a to pivotally connect the actuator 110 to the housing 102.

Pusher tube 150, positioned within elongated tube 104, includes a collar 152 having link engagement pins 154 configured for reception in elongated slots 127a of link 122. Block 143, which has a valve mechanism therein, is fixed about pusher tube 150 and has a lumen formed therein to accommodate sliding movement of pusher tube 150. Valve block 143 includes an opening 145 configured and dimensioned to receive a tube 162 in fluid communication with fluid container 160.

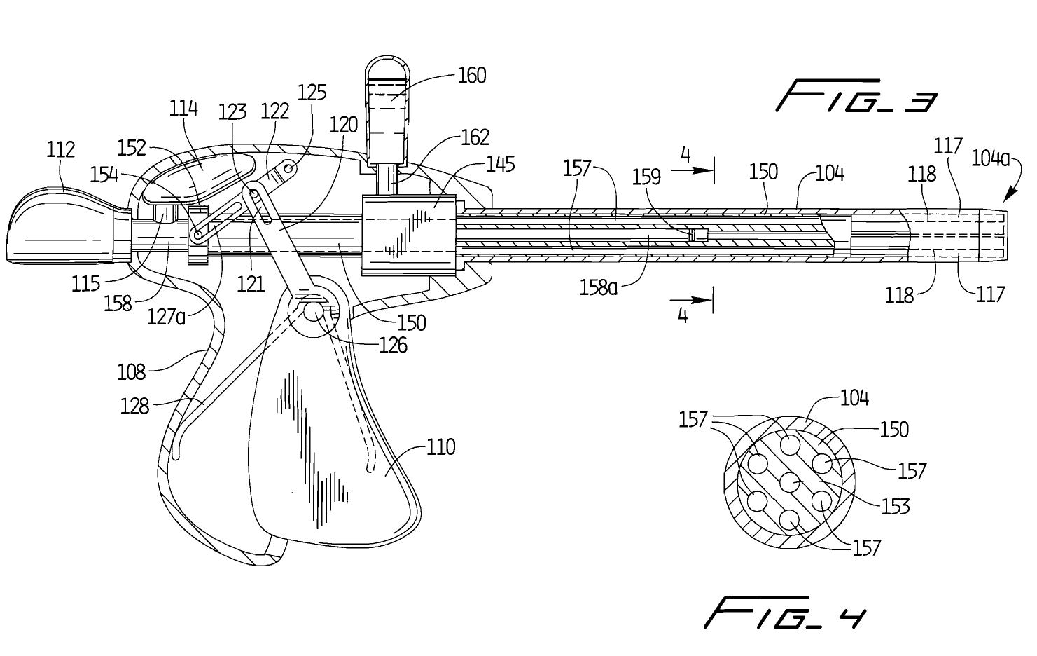

A vacuum chamber 114 is in communication with vacuum tube 115 which extends into and is in fluid communication with the internal lumen of elongated longitudinally extending vacuum tube 158. Elongated vacuum tube 158 has a reduced diameter region 158a with an O-ring seal 159. The vacuum tube 158 extends through a central lumen 153 in the pusher tube 150. As shown in FIG. 3, vacuum chamber 114 is preferably positioned within a proximal region of the housing 102, internally thereof. However, it can alternatively be positioned within other regions of the housing 102 and can also be positioned external of the housing 102 with the vacuum tube extending through the housing wall to communicate with the tube 158 and/or tube 115.

The pusher tube 150 has a series of longitudinally extending lumens 157 formed therein, preferably corresponding to the number of retractor arms of the retractor assembly. As shown lumens 157 are positioned radially of central lumen 153. Retractor arms or elongate members 118 have lumens 117 formed therein, each terminating in a distal opening 119 (only a few of which are labeled for clarity). Lumens 117 are in fluid communication with the lumens 157 of the pusher tube 150. The retractor members 118 are attached to the pusher tube 150 such that sliding movement of the pusher tube 150 slides the retractor members 118.

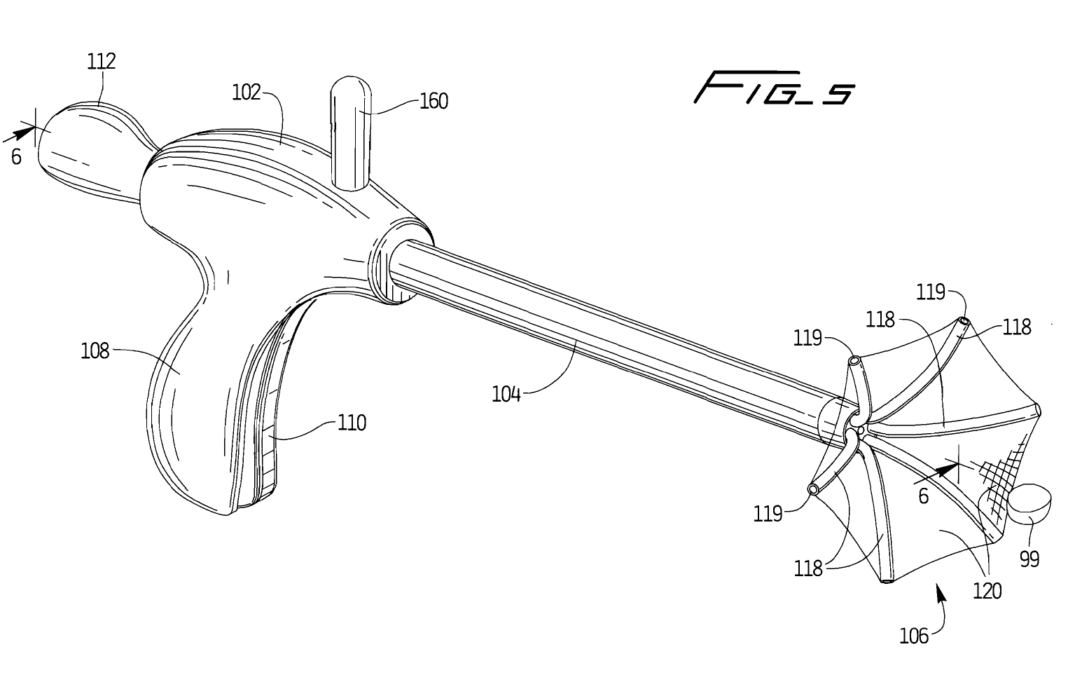

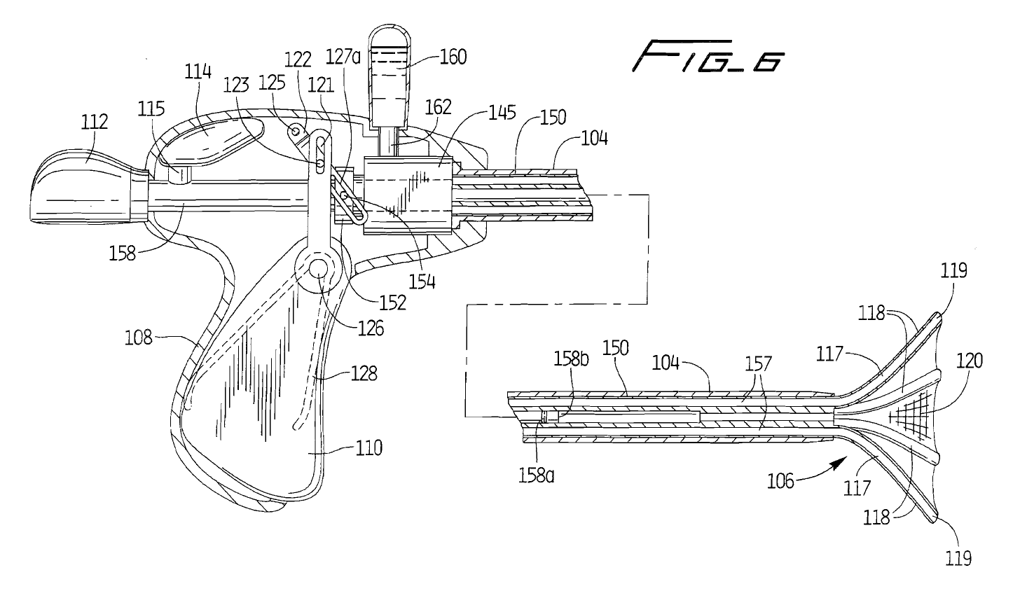

With reference to FIGS. 5-6, the retractor 106 can have one or more webbing elements 120 positioned between the retractor arms 118. Alternatively, no webbing element 120 or webbing only between some of the elongate retractor members 118 can be provided. A plurality of webbing elements 120 alternatively sandwiched between at least two elongate members 118 is also contemplated. A single webbing element between multiple or all the elongated elements is also contemplated. The webbing element 120 can be mesh, solid or other form appreciated by one skilled in the art. The webbing element 120 and each elongate member 118 are configured and dimensioned to collapse as withdrawn within the outer tube 104 for insertion through an access port (not shown), and then expand (spread) inside the body cavity when advanced from the outer tube 104 as the retractor members 118 expand. To withdraw the device after the procedure, the retractor 106 is withdrawn back inside tube 104. The retractor members 118 with attached web can optionally articulate into and out of various grasping/retracting orientations. The retractor 106 can be used to manipulate or dissect tissue.

The vacuum chamber 114 suctions the specimen 99 (shown schematically) through the distal end 104a of shaft 104 in the space between the retractor members 118 through the central lumen 157 of pusher tube 150, into the distal opening 158b of tube 158 (seated within central lumen 157) and through the lumen of pusher tube 158 into the tank or container 112. The container 112 can include a removable cover to remove the specimen for pathology, if necessary.

As noted above, it is envisioned that in certain embodiments at least one, and preferably several or even all, of the plurality of elongate members 118 has an aperture 119 formed on the distal end and a lumen 117 defined therethrough. Container 160 is disposed in fluid communication with the retractor members 118 for storing sealants, adhesives, medicaments, bio active agents, or the like, hereinafter collectively referred to as “fluids.” These fluids can be applied to various tissues, e.g. frangible tissues that are highly vascular and bleed easy, or lung tissue which is very delicate. The fluids can also be stored “in form” for direct application, or in “dual form”, such that combinations of the various fluids provide a suitable medical mixture. The container 160 is shown extending transversely from the housing 102 but alternatively can be positioned at other regions of the housing 102 and at other orientations. It could also be positioned within the housing 102.

Delivery tube 162 is in fluid communication with the container 160 which communicates with pusher tube 150 to supply fluid through the lumens 157 and into the lumens 117 of the retractor elements 118 to exit out of distal openings 119 into the patient's body. Note, alternatively or in addition, side holes can be provided in the retractor elements 118 for fluid delivery. Thus, fluids can travel from the container 160, through the delivery tube 162, into the pusher tube 150, through the lumens 157 formed therein and through the lumens 117 of the respective elongate member 118. The fluid flow continues through the lumens 117 in the elongate member(s) 118 and exits distal opening 119 at the distal end of the elongate member 118. In this manner, fluid communication is achieved between the container 160 and the distal end of the elongate members 118. In some embodiments, one or more of the elongate members 118 can have a nozzle attached to the distal end thereof, enabling the fluid to be delivered in a spray.

To advance the retractor elements 118, actuator handle 110 is pulled proximally toward handle 108, causing yoke 120 to pivot about pivot pin 126 from the position of FIG. 3 to the position of FIG. 6. As yoke 120 pivots it causes link 122 pivot about connecting pin 125 due to the engagement of transverse pins 123 with slots 121 of yoke 120. Such pivoting of link 122 forces pusher tube 150 distally due to the engagement of slots 127a with link engagement pin 154 of collar 152. The distal movement of pusher tube 150 from the position of FIG. 3 to the position of FIG. 6 moves the retractor members 118 distally to expose them from tube 104 so they can move to the spread or expanded position. The retractor elements can be made of a shape memory material with a shape memorized expanded position and/or the web can be made of a material which expands to spread the retractor elements 118.

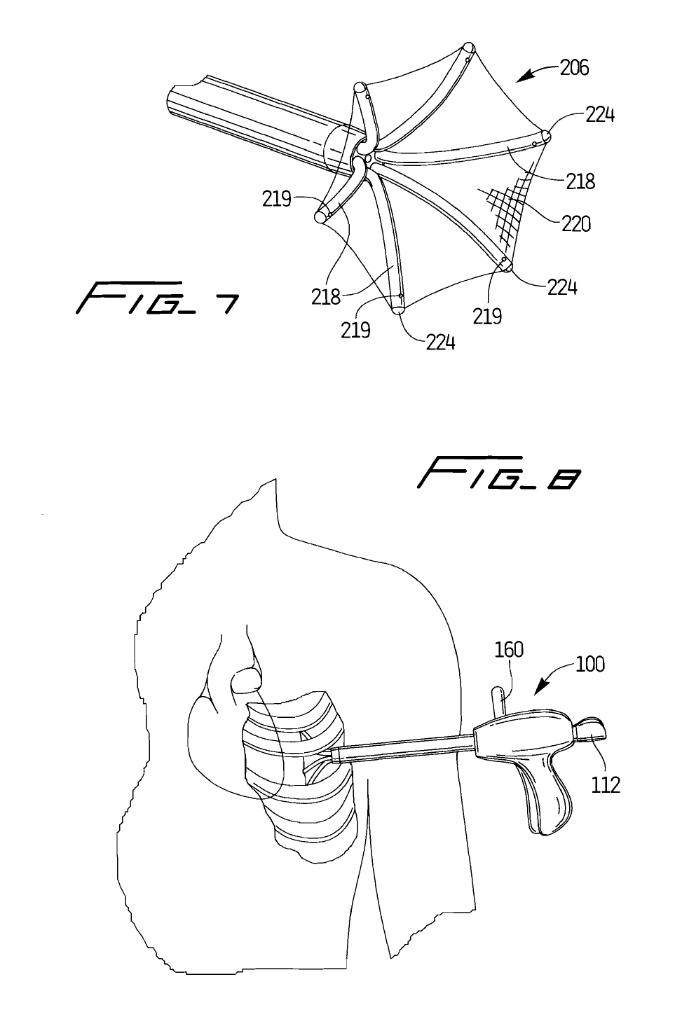

In an alternate embodiment of FIG. 7, the retractor 206 has one or more sensors 224 (only some of which are labeled for clarity) attached to the retractor elements 218 thereof for facilitating diagnostics. The sensors 224 can be disposed on the distal end of one or more of the elongate members 218 or at the distal end of one or more nozzles. Other locations are also contemplated. The sensors or other diagnostic devices can also be used for visualization of tissue type (cancer or no cancer, normal or abnormal tissue) or true diagnosis (the cells that are adenocarcinoma that are penetrated through the entire thickness of the tissue) or other similar approaches. The elongate members 218 can include one or more side openings 219 for fluid flow or alternatively, the sensors can be positioned proximal of the distal tip and the distal tip can have distal openings as in the embodiment of FIGS. 1-6. Webbing 220 similar to webbing 120 of FIG. 6 is preferably fixed to the elongate members 218. Variations on the webbing discussed above with respect to the embodiment of FIGS. 1-6 are applicable to the embodiment of FIG. 7 as well.

FIG. 8 illustrates the device 100 being inserted through the patient's ribs for a minimally invasive thoracic procedure by way of example. The device 100 is preferably inserted through a thoracoport (not shown) to enter the space between the ribs and access the surgical site within the thoracic cavity.

With respect to fluids being delivered/discussed above, it is contemplated that the fluid can be a biocompatible adhesive, including, and not limited to, adhesives which cure upon tissue contact, which cure upon exposure to ultraviolet (UV) light, which are two-part systems which are kept isolated from one another and cure upon coming into contact with one another, which are pressure sensitive, which are any combinations thereof, or any other known suitable adhesive. In one embodiment, an adhesive having a cure time of from about 10 to 15 seconds may be used. In another embodiment, an adhesive having a cure time of about 30 seconds may be used. Adhesives with other cure times are also contemplated.

The container 160 may be impregnated with a pre-cured adhesive or sealant. The pre-cured sealant or adhesive will react with the moisture and/or heat of the body tissue to thereby activate the sealing and/or adhesive properties of the sealant or adhesive. It is envisioned that the pre-cured sealant or adhesive may be a hydro-gel or the like.

Other surgically biocompatible wound treatment materials which may be employed in or applied by the device 100 include adhesives whose function is to attach or hold organs, tissues or structures, sealants to prevent fluid leakage, hemostats to halt or prevent bleeding, and medicaments.

Examples of adhesives which can be employed include protein derived, aldehyde-based adhesive materials, for example, the commercially available albumin/glutaraldehyde materials sold under the trade designation BioGlue™ by Cryolife, Inc., and cyanoacrylate-based materials sold under the trade designations Indermil™ and Derma Bond™ by Tyco Healthcare Group, LP and Ethicon Endosurgery, Inc., respectively.

Examples of sealants, which can be employed, include fibrin sealants and collagen-based and synthetic polymer-based tissue sealants. Examples of commercially available sealants are synthetic polyethylene glycol-based, hydrogel materials sold under the trade designation CoSeal™ by Cohesion Technologies and Baxter International, Inc.

Examples of hemostat materials, which can be employed, include fibrin-based, collagen-based, oxidized regenerated cellulose-based and gelatin-based topical hemostats. Examples of commercially available hemostat materials are fibrinogen-thrombin combination materials sold under the trade designations CoStasis™ by Tyco Healthcare Group, LP, and Tisseel™ sold by Baxter International, Inc. Hemostats herein include astringents, e.g., aluminum sulfate, and coagulants.

The medicament may also be disposed or impregnated into the container 160. The medicament may include one or more medically and/or surgically useful substances such as drugs, enzymes, growth factors, peptides, proteins, dyes, diagnostic agents or hemostasis agents or any other pharmaceutical used in the prevention of stenosis.

The bioactive agent may be selected from any bioactive agent known to those skilled in the art. Suitable bioactive agents include, but are not limited to, drugs, such as antiseptics, anesthetics, muscle relaxants, antihistamines, decongestants, antimicrobial agents, anti-viral agents, anti-fungal agents, antimalarials, amebicides, antituberculosal agents, antiretroviral agents, leprostatics, antiprotazoals, antihelmitics, antibacterial agents, steroids, hematopoietic agents, antiplatelet agents, anticoagulants, coagulants, thrombolytic agents, hemorrheologic agents, hemostatics, plasma expanders, hormones, sex hormones, uterine-active agents, bisphosphonates, antidiabetic agents, glucose-elevating agents, growth hormones, thyroid hormones, inotropic agents, antiarrhythmic agents, calcium channel blockers, vasodilators, sympatholytics, antihyperlipidemic agents, vasopressors, angiotensin antagonists, sclerosing agents, anti-impotence agents, urinary alkanizers, urinary acidifiers, anticholinergics, diuretics, bronchodilators, surfactants, antidepressants, antipsychotics, antianxiety agents, sedatives, hypnotics, barbiturates, antiemetic agents, analgesics, stimulants, anticonvulsants, antiparkinson agents, proton pump inhibitors, H₂-antagonists, antispasmodics, laxatives, antidiarrheals, antiflatulents, digestive enzymes, gallstone solubilizing agents, antihypertensive agents, cholesterol-lowering agents, radiopaque agents, immune globulins, monoclonal antibodies, antibodies, antitoxins, antivenins, immunologic agents, anti-inflammatory agents, antineoplastic agents, alkylating agents, antimetabolites, antimitotic agents, radiopharmaceuticals, vitamins, herbs, trace elements, amino acids, enzymes, chelating agents, immunomodulatory agents and immunosuppressive agentsmuscle; coating materials such as lubricants; diluents, such as sterile saline, sterile dextrose in water and sterile lactated ringers; wound healing agents, adhesives, sealants, blood products, blood components, preservatives, colorants, dyes, ultraviolet absorbers, ultraviolet stabilizers, photochromic agents, proteins, polysaccharides, peptides, genetic material, immunological agents, anti-colonization agents, diagnostic agents, imaging agents and combinations thereof.

It will be understood that various modifications may be made to the embodiments disclosed herein. Therefore, the above description should not be construed as limiting, but merely as an exemplification of the embodiments. Those skilled in the art will envision other modifications within the scope and spirit of the present disclosure. Such modification and variations are intended to come within the scope of the following claims.

What is claimed is:

1. A surgical device comprising a housing, an elongated shaft extending distally from the housing, a retractor including a plurality of retractor elements extending distally from the elongated shaft, a vacuum tube positioned within the elongated shaft, a vacuum chamber in communication with the vacuum tube for applying a suction force to remove a specimen from a patient's body, and a fluid container to deliver a fluid to the body.

2. The surgical device of claim 2, wherein at least one of the retractor elements includes a lumen extending therethrough, the lumen communicating with the fluid container to apply a fluid to a surgical site.

3. The surgical device of claim 2, wherein the lumen of the retractor elements has a distal opening communicating with the lumen.

4. The surgical device of claim 2, wherein the fluid container is positioned external of the housing.

5. The surgical device of claim 1, further comprising a specimen container to receive a specimen removed from the body, the specimen container being in communication with the vacuum tube.

6. The surgical device of claim 1, further comprising an actuator for moving the retractor elements distally from a first collapsed position within the elongated shaft to a second expanded position distal of the shaft.

7. The surgical device of claim 1, wherein the vacuum chamber is positioned inside of the housing.

8. The surgical device of claim 7, wherein the specimen container is positioned external of the housing.

9. The surgical device of claim 8, wherein the fluid container is positioned external of the housing.

10. The surgical device of claim 1, wherein the specimen container is positioned external of the housing.

11. The surgical device of claim 1, wherein the retractor elements are biased to an open position.

12. The surgical device of claim 1, further comprising a web extending between at least two of the retractor elements.

13. The surgical device of claim 1, wherein at least one of the retractor elements includes a sensor.

14. The surgical device of claim 6, wherein the actuator includes a pusher tube, wherein the vacuum tube is positioned within the pusher tube, and the pusher tube includes a plurality of lumens communicating with the fluid container to deliver a fluid to the retractor elements.

15. A surgical device comprising a housing, an elongated shaft extending distally from the housing, a vacuum tube positioned within the elongated shaft, a vacuum chamber in communication with the vacuum tube for applying a suction force to remove a specimen from a patient's body, a fluid container to deliver a fluid to the body, and a specimen container to receive a specimen removed from the body, the specimen container being in communication with the vacuum tube.

16. The surgical device of claim 15, wherein the vacuum chamber is positioned inside of the housing.

17. The surgical device of claim 16, wherein the specimen container is positioned external of the housing.

18. The surgical device of claim 17, wherein the fluid container is positioned external of the housing.
